(19) United States
(12) Patent Application Publication
(10) Pub. No.: US 2026/0315120 A1
(43) Pub. Date: Oct. 15, 2026

(54) OSTEOSYNTHESIS PLATE AND METHOD FOR MANUFACTURING AN OSTEOSYNTHESIS PLATE

(71) Applicant: Karl Leibinger Asset Management GmbH & Co. KG, Mühlheim an der Donau (DE)

(72) Inventors: Martin Pabst, Reichenau (DE); Iris Zschocke, Haslach im Kinzigtal (DE); Harald Merkt, Blumberg Baden (DE); Vincent Pfeiffer, Mühlheim an der Donau (DE); Klaus Kohler, Hausen (DE); Martin Meer, Vöhringen (DE)

(21) Appl. No.: 19/463,597

(22) Filed: Jan. 29, 2026

(30) Foreign Application Priority Data
Jan. 30, 2025 (DE) 102025103444.3

(51) Int. Cl. A61B 17/80; A61B 17/00

(52) U.S. Cl. CPC A61B 17/80; A61B 2017/00526

(57) ABSTRACT

An osteosynthesis plate (P) for fixation of bone fragments includes a main body (G) with a top side (G1) and an underside (G2). The underside (G2) is configured for placement of the osteosynthesis plate (P) on the bone fragments. The osteosynthesis plate (P) includes a plurality of openings (A) formed between the top side (G1) and the underside (G2) and configured for holding screws (SK). The main body (G) has a multilayer design and includes a core layer (K), a first cover layer (S1) that at least partially covers the core layer (K) on a first side, and a second cover layer (S2) that at least partially covers the core layer (K) on a second side disposed opposite the first side. The core layer (K) is formed from a first material, and the first and second cover layers (S1, S2) are formed from a second material. The first material differs from the second material, and the first material has higher ductility than the second material.

### CROSS-REFERENCE TO RELATED APPLICATION

The present application is related and has right of priority to German Patent Application No. DE 102025103444.3 filed on Jan. 30, 2025, which is incorporated by reference in its entirety for all purposes.

### TECHNICAL FIELD

The invention relates generally to an osteosynthesis plate for the fixation of bone fragments, and to a method for manufacturing such an osteosynthesis plate.

### BACKGROUND

In osteosynthesis, after a bone fracture, two or more bone fragments are connected to one another within the framework of a surgical procedure in order to enable the bone fragments to grow together. The goal of osteosynthesis is to achieve stable fixation of the bone fragments, wherein this fixation is to take place with the bone fragments in a correct position with respect to one another and thus, optionally, with correction of malalignments. In addition to a fixation using wire or screws, osteosynthesis plates are also used depending on the area of application, wherein the osteosynthesis plate is placed on the bone in the region of the fracture and attached to each of the bone fragments to be connected to one another. Attachment is usually carried out using screws, for the purpose of which a plurality of through-holes is then formed in the osteosynthesis plate.

In osteosynthesis plates of this type, different biocompatible materials are used, for example, titanium, titanium alloys, stainless steel, or biocompatible polymers, such as polyether ether ketone (PEEK). In selecting the material, in addition to the required strength and biocompatibility, the cooperation with the fastening screws is of great significance. For example, EP 2 956 073 A1 teaches a pressure plate made of pure titanium between grade 0 and grade 3, such that the pressure plate is formed from a softer material than a bone screw made of the titanium alloy Ti-Al6-V4 and cooperating therewith. Due to a material combination of this type, the bone screw can deform the pressure plate, such that an engagement between the screw head and the pressure plate is improved. If greater stability of the pressure plate is required, the pressure plate and the bone screw can be made of the titanium alloy Ti-Al6-V4, wherein inlays made of pure titanium are inserted in the pressure plate body for holding the bone screws.

Inserting inlays of this type into a base plate in a reliable manner is complicated, however. This is the case because the inlay must not become twisted when the base plate is screwed, such that a rotation lock is required. The inlay must also be prevented from being pressed out of the base plate in the screwing direction by a suitable structural design.

### BRIEF SUMMARY

Example aspects of the invention provide an osteosynthesis plate, which is distinguished by high stability and nevertheless enables a thread to be cut during screwing, and which can be easily and reliably manufactured. Example aspects of the invention also provide a method for manufacturing an osteosynthesis plate of this type.

An osteosynthesis plate for the fixation of bone fragments is provided, which osteosynthesis plate has a main body with a top side and with an underside opposite the top side. The underside is intended for the placement of the osteosynthesis plate on the bone fragments. The osteosynthesis plate has a plurality of openings which extend between the top side and the underside. By screwing one screw through each opening of this type, the osteosynthesis plate can be attached to the bone.

According to example aspects of the invention, the main body has a multilayer design including a core layer, a first cover layer at least partially covering the core layer, and a second cover layer at least partially covering the core layer. The first cover layer is arranged on a first side of the core layer. The second cover layer is arranged on a second side of the core layer, which is opposite the first side. The core layer is made of a first material, whereas the two cover layers are formed from a second material which differs from the first material. The first material has higher ductility than the second material. In other words, the resistance of the core layer to plastic deformation is lower than the resistance of the cover layers to plastic deformation. “Higher ductility” is preferably understood within the framework of the this document to mean that the tensile strength of the less ductile material is greater than the tensile strength of the more ductile material by at least the factor 1.5.

Due to the design according to example aspects of the invention, the osteosynthesis plate has a relatively soft, ductile core, whereas the cover layers are formed from a material that is harder and less ductile in comparison to the core. As a result, during a flexural loading of the osteosynthesis plate in the state in which the osteosynthesis plate is screwed together with the bone, high stability is ensured without the need to dispense with the screwing advantages of the soft core. Due to the layered design, during the manufacture of the osteosynthesis plate, there is no need to laboriously create joining regions for the insertion of inlays. A rotation lock and securing the inlays against being pressed out axially can also be dispensed with.

According to a first possible example variant, the core layer is formed from pure titanium, for example from grade 1 titanium, grade 2 titanium, grade 3 titanium, or grade 4 titanium, wherein grade 2 titanium is particularly preferred. The first and the second cover layer are formed, in this first example variant, from a titanium alloy, for example from Ti-6Al-4V or from Ti-Al6-Nb7. Alternatively, the first and the second cover layer in the first example variant can be formed from steel, in particular from a rust- or acid-resistant steel grade, for example from X2CrNiMo18-15-3, or from X2CrMnMo 18-13-3, or from X4CrNiMnMo 21-9-4, or from CoCr28Mo6 having a carbon content greater than 0.2%.

For attaching an osteosynthesis plate according to this first possible example variant to the bone, screws made of Ti-6Al-4V, of X2CrNiMo18-15-3, or of X2CrMnMo 18-13-3 are preferably used. These screw materials have greater strength than the core layer, such that the screws can cut a thread into the core layer or deform an existing thread in the core layer. As a result, the hold of the screw can be improved. By combining an osteosynthesis plate and a plurality of screws, a set can be formed.

According to a second possible example variant, the core layer is formed from a first steel grade, whereas the first and the second cover layer are formed from a second steel grade or from a titanium alloy. The first steel grade according to the second example variant can be formed, for example, from CoCr28Mo6 having a carbon content less than 0.055%, or from X2CrNiMo18-15-3, or from X4CrNiMnMo 21-9-4, or from X2CrMnMo 18-13-3. The second steel grade according to the second example variant can be formed from CoCr28Mo6 having a carbon content greater than 0.2%, or from X2CrNiMo18-15-3, or from X4CrNiMnMo 21-9-4, or from X2CrMnMo 18-13-3. The titanium alloy for the cover layers according to the second example variant can be formed, for example, from Ti-Al6-Nb7 or from Ti-6Al-4V. It is important to note that the tensile strength of these materials depends on whether the materials are in the strain-hardened or annealed state. With respect to the particular combination of materials for the core layer and the cover layers, it is therefore important to make certain that the materials have a suitable ductility difference in the finished state of the osteosynthesis plate.

For attaching an osteosynthesis plate according to this second possible example variant to the bone, screws are preferably selected from the following materials: Ti-6Al-4V, X2CrNiMo18-15-3, or X2CrMnMo 18-13-3 with a core layer of the osteosynthesis plate made of CoCr28Mo6 having a carbon content less than 0.055%;
X2CrMnMo 18-13-3 with a core layer of the osteosynthesis plate made of X2CrNiMo18-15-3;
X2CrNiMo18-15-3 with a core layer of the osteosynthesis plate made of X2CrMnMo 18-13-3.

These screw materials, given suitable heat treatment and/or strain hardening, have greater strength than the core layer, such that the screws can cut a thread into the core layer or deform an existing thread in the core layer. As a result, the hold of the screw can be improved. By combining an osteosynthesis plate and a plurality of screws, a set can be formed.

Preferably, a shoulder for supporting the screw is formed in at least one of the openings, wherein this shoulder is arranged in the core layer. Due to an example embodiment of this type, the screw can be supported in the core layer while cutting a thread in the core layer. This improves the hold of the screw and offers the greatest possible freedom in selecting the angle between the screw and the osteosynthesis plate.

Preferably, an internal thread is formed in at least one of the openings. The internal thread is formed either exclusively or predominantly in the core layer and is designed to cooperate with an external thread of the screw. If the internal thread is not completely formed in the core layer, then preferably only a short inlet or outlet of a thread turn is arranged in one of the cover layers. Complete thread turns are arranged exclusively in the core layer.

Preferably, the core layer, the first cover layer, and/or the second cover layer has a constant layer thickness. An example embodiment in which the first cover layer has the same layer thickness as the second cover layer is particularly preferred. Such an example embodiment is advantageous in particular when the cover layers do not need to be ablated during the production of the osteosynthesis plate in order to achieve the desired layer thickness. This is the case because, due to a symmetrical layered design of this type, an inadvertent mix-up of the underside and the top side during the manufacture can be ruled out.

Alternatively, it can be provided that the first cover layer and/or the second cover layer has a non-constant layer thickness. An example embodiment of this type can be advantageous, for example, when the osteosynthesis plate has a significantly different loading in one bending direction than in the other bending direction, such that a geometry can be selected that is optimized with respect to loading. An example embodiment of this type is also advantageous when the osteosynthesis plate is to be plastically deformed at a certain section by the surgeon. In this section, the osteosynthesis plate can have a smaller layer thickness of the cover layer or can be locally uncoated.

According to a first possible example embodiment, the first cover layer can be formed exclusively on the top side, and the second cover layer can be formed exclusively on the underside of the osteosynthesis plate. This is the case because the osteosynthesis plate, when in the implanted state, has the greatest tensile stresses and compressive stresses on the top side and the underside in numerous applications, such that a geometry that is optimized with respect to loading is formed due to the design of the cover layers on the top side and the underside, which are harder in comparison to the core layer.

Preferably, the first cover layer and/or the second cover layer covers the entire surface of the core layer on the top side and/or on the underside, respectively, of the osteosynthesis plate. Excepted from such a coverage over the entire surface are openings between the top side and the underside, and holding and positioning geometries formed on one side, such as spherical caps or blind holes. Chamfers at the transitions between the surfaces and suture holes at the edges are also excepted therefrom. A formation of the cover layer over the entire surface of the top side and the underside is therefore understood to mean that smooth, non-function-forming surfaces of the top side and the underside are covered by the first cover layer and the second cover layer, respectively.

In an example embodiment of the first and the second cover layer on the top side and on the underside, respectively, of the osteosynthesis plate, the layer thickness of the first and the second cover layer is preferably between two-tenths (0.2) millimeter and two (2) millimeters, particularly preferably between two-tenths (0.2) millimeter and one (1) millimeter. Such a dimensioning of the cover layers has been found to be advantageous.

According to a further possible example embodiment, the first and/or the second cover layer covers the core layer only in sections, such that the core layer is partially exposed on the top side and/or on the underside, respectively. An example embodiment of this type is advantageous when the osteosynthesis plate is to be plastically deformed at a certain section by the surgeon. If the osteosynthesis plate has, for example, a design with a head region and a shaft region, wherein the head region is designed to be attached to a metaphysis, and the shaft region is designed to be attached to a diaphysis, the shaft region could be designed without a cover layer at least in some sections, in order to be able to more easily adapt the osteosynthesis plate in this region to the individual anatomy of the patient.

Preferably, the core layer has a layer thickness that is at least one and a half (1.5) times, particularly preferably three (3) times, as great as the first and the second cover layer at their thickest point. Due to such an example embodiment, an osteosynthesis plate is provided, which has a thin and comparatively hard layer on the top side and the underside, which can support the high tensile stresses and compressive stresses without plastic deformation, and which has a thick and comparatively soft core, which is well suited for the cutting of a thread. Particularly preferred relationships of the layer thicknesses of the core layer and the cover layers are: one-tenth (0.1) of the overall thickness of the osteosynthesis plate as the layer thickness of each individual cover layer and eight-tenths (0.8) of the overall thickness of the osteosynthesis plate as the layer thickness of the core layer; or one-twentieth (0.05) of the overall thickness of the osteosynthesis plate as the layer thickness of each individual cover layer, and eighteen-twentieths (0.9) of the overall thickness of the osteosynthesis plate as the layer thickness of the core layer.

In an example embodiment of this type, the shoulder, which arranged in the core layer, for supporting the screw can be arranged obliquely to a direction of extent of the core layer. Thus, a pre-alignment of the screw which deviates from an orthogonal direction proceeding from the underside of the osteosynthesis plate can be made possible. Due to such an example design, the osteosynthesis plate can be used for fractures in which a pre-alignment of this type is highly significant, such as fractures in the region of the metaphysis or of the epiphysis of a tubular bone.

According to an alternative example embodiment, the core layer has a layer thickness that is either less than or at most one and a half (1.5) times as great as the first and the second cover layer at its thickest point. As an exemplary limit for a core layer having a smaller layer thickness than the cover layers, a relationship can be considered, in which the core layer has a layer thickness of one-fifth (0.2) of the overall thickness of the osteosynthesis plate, and each of the cover layers has a layer thickness of two-fifths (0.4) of the overall thickness of the osteosynthesis plate. Such an example embodiment is advantageous in particular with thin-walled plates, such as those that are used in surgery of the mouth, jaw, and face, for rib fractures, or for fractures of pelvic bones. Such an embodiment is suitable in particular in a design having a constant layer thickness of the core layer and of the two cover layers.

In an example embodiment of this type, the shoulder, which arranged in the core layer, for supporting the screw is preferably arranged parallel to a direction of extent of the core layer. A pre-alignment of the screw receptacle that deviates from an orthogonal direction proceeding from the underside of the osteosynthesis plate is therefore not given in a design of this type. This is frequently not required, however, with thin-walled osteosynthesis plates. Therefore, the thread can be cut exclusively or predominantly in the core layer, also in an example embodiment of this type, such that a reliable hold of the screw is ensured.

According to a further possible example embodiment, the first cover layer and the second cover layer can extend along opposing lateral faces of the osteosynthesis plate, such that the core layer, the first cover layer, and the second cover layer extend from the top side to the underside. In other words, the first and the second cover layer in this example embodiment are formed on the side edges and not on the top side and the underside of the osteosynthesis plate. In an example embodiment of this type, the layer thickness of the first and/or the second cover layer is preferably between a half (0.5) millimeter and five (5) millimeters.

Preferably, the osteosynthesis plate has an elongate, non-planar shape. Due to an example design of this type, the osteosynthesis plate is particularly well adapted to the anatomy of the bone. The layer-like design can also be applied in a non-planar shape of this type, as is made clear with the following comments regarding the manufacturing methods.

A method is provided for manufacturing an osteosynthesis plate, which method includes the following steps. In a first step, a planar raw material is provided, which has the core layer as well as the first and the second cover layer. A raw material of this type can be manufactured in different ways, for example, by rolling or roll-bonding. In a second step, an intermediate stage of the osteosynthesis plate is separated from the raw material. In a third step, shaping is carried out in order to obtain a desired non-planar shape of the osteosynthesis plate. The non-planar shape is then heat-treated in a fourth step. In an optional additional step, a surface treatment and/or a surface coating of the heat-treated osteosynthesis plate is carried out.

The separation from the raw material, which is carried out in the second step, can take place in different ways, for example by water-jet cutting, laser cutting, punching, or milling. The shaping carried out in the third step can also be carried out in different ways, for example, by cold-forming or hot-forming. The surface treatment, which is optionally carried out, can take place, for example, by machining or by abrasive blasting. The optional surface coating can be, for example, anodization.

The heat treatment carried out in the fourth step can be, for example, solution annealing, precipitation annealing, or sub-critical annealing. According to a first possible example design variant, in the fourth step, a solution annealing and/or a sub-critical annealing can be carried out at a temperature of up to nine hundred degrees Celsius (900° C.), for example under an argon protective gas. Then, water cooling and/or press hardening is carried out, and ageing is carried out at approximately five hundred degrees Celsius (500° C.) for a time duration of four to eight (8) hours. According to a second possible example design variant, solution annealing is carried out at a temperature of approximately one thousand and twenty degrees Celsius (1020° C.), for example under an argon protective gas. Then, simple cooling is carried out. Ageing does not need to be carried out after the solution annealing in this second example design variant.

Preferably, the second step (separation) is carried out prior to the third step (shaping), such that the intermediate stage separated from the raw material has a planar shape. Alternatively, it is also conceivable that the second step is carried out after the third step, such that the intermediate stage separated from the raw material already has the desired non-planar shape. Due to the non-planar shape, the osteosynthesis plate is particularly well adapted to the anatomy of the bone, such that an additional shaping by the surgeon during the placement of the osteosynthesis plate on the bone during surgery is either not required or is required only to a small extent.

Preferably, the manufacturing method includes, in a fifth step, machining for creating a geometry of the openings in the osteosynthesis plate. In this fifth step, in addition to the openings, further geometries can also be produced, for example, suture anchors or a thread for attaching a drill-guide block to the osteosynthesis plate. The fifth step is carried out either immediately after the second step (separation), after the third step (shaping), or after the fourth step (heat-treating).

The described steps of the manufacturing method are not final, such that further manufacturing steps can be included. For example, rounding-off, deburring, or grinding can be provided for creating a surface that is suitable for the surgical application. For the sake of clarity, these steps are not explicitly mentioned, since a person skilled in the art will use steps of this type at the suitable point in the manufacturing method.

### BRIEF DESCRIPTION OF THE DRAWINGS

Exemplary embodiments of the invention are described in detail with reference to the figures, wherein:

FIG. 1 and FIG. 2 each show a view of an osteosynthesis plate according to a first exemplary embodiment;

FIG. 3 and FIG. 4 each show a sectional view of the osteosynthesis plate according to the first exemplary embodiment;

FIG. 5 shows a sectional view of an osteosynthesis plate according to a second exemplary embodiment;

FIG. 6 and FIG. 7 each show a view of an osteosynthesis plate according to a third exemplary embodiment;

FIG. 8 and FIG. 13 show different views of intermediate stages during the manufacture of an osteosynthesis plate according to the first exemplary embodiment,

FIG. 14 through FIG. 22 show different views of intermediate stages during the manufacture of an osteosynthesis plate according to the third exemplary embodiment,

FIG. 23 shows a view of an osteosynthesis plate with screws arranged therein;

FIG. 24 through FIG. 26 each show a view of an osteosynthesis plate according to a fourth exemplary embodiment;

FIG. 27 through FIG. 29 each show a view of an osteosynthesis plate according to a fifth exemplary embodiment;

FIG. 30 shows a view of an osteosynthesis plate according to a sixth exemplary embodiment;

FIG. 31 and FIG. 32 each show a view of an osteosynthesis plate according to a seventh exemplary embodiment;

FIG. 33 and FIG. 34 each show a view of an osteosynthesis plate according to an eighth exemplary embodiment; and

FIG. 35 through FIG. 37 each show a view of an osteosynthesis plate according to a ninth exemplary embodiment.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

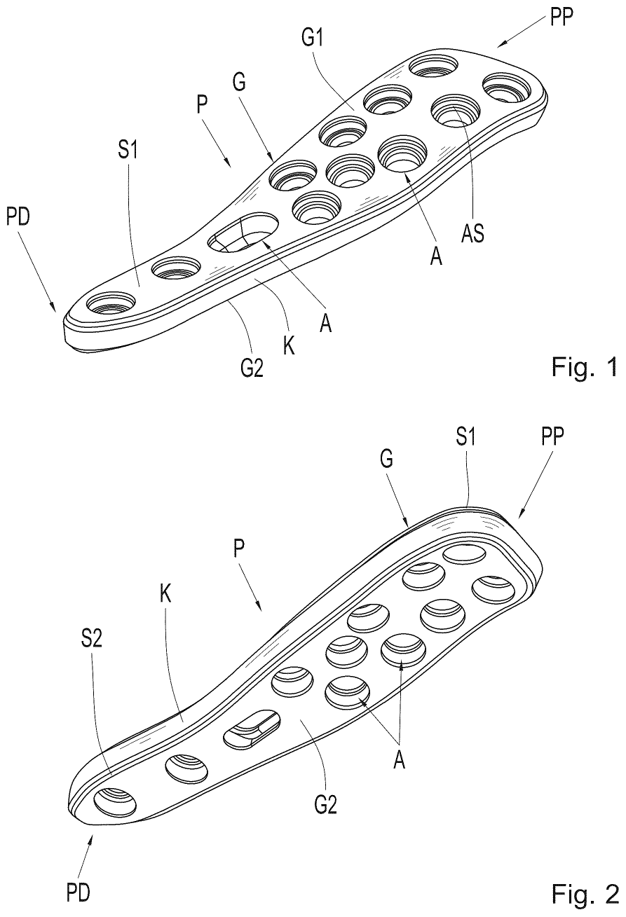

FIG. 1 and FIG. 2 each show an isometric view of an osteosynthesis plate P according to a first exemplary embodiment. The osteosynthesis plate P has a main body G with a top side G1 and with an underside G2. The underside G2 is intended for the placement of the osteosynthesis plate P on bone fragments, which are not represented in the figures. The osteosynthesis plate P has a plurality of openings A, which penetrate the osteosynthesis plate P from the top side G1 to the underside G2. The openings A are designed to hold one screw each, which is not represented in FIG. 1 and FIG. 2. By the screws, the osteosynthesis plate P is attached to the bone fragments. To this end, at least one of the openings A has a shoulder AS, against which a section of the screw can be supported. The exemplary embodiment represented in FIG. 1 and FIG. 2 shows an osteosynthesis plate P for application on the proximal humerus, having a proximal end PP and a distal end PD. The osteosynthesis plate P has an elongate and curved, non-planar shape in order to be adapted to the anatomy of a bone in the best way possible, in this case, by way of example, for the shape of the proximal humerus.

The main body G of the osteosynthesis plate P has a multilayer design which includes a core layer K as well as a first and a second cover layer S1, S2. The first cover layer S1 covers the core layer K on the top side G1. The second cover layer S2 covers the core layer K on the underside G2. The core layer K is formed from pure titanium, for example from grade 2 titanium. The first and the second cover layer S1, S2 are formed from a titanium alloy, for example from Ti-6Al-4V. Alternatively, the first and the second cover layer S1, S2 could be formed from steel, for example from X2CrNiMo18-15-3 or from X4CrNiMnMo 21-9-4. The first and the second cover layer S1, S2 have, in the first exemplary embodiment, an identical, constant layer thickness, which is approximately four-tenths millimeter (0.4 mm) in the present example. The core layer K is significantly thicker in comparison to the cover layers S1, S2 and, in the present example, has a thickness of approximately three and two-tenths millimeters (3.2 mm).

According to an alternative example embodiment, the core layer K is formed from the steel grade CoCr28Mo6 having a carbon content less than 0.055%, whereas the two cover layers S1, S2 are formed from the steel grade CoCr28Mo6 having a carbon content greater than 0.2%. According to a second alternative example embodiment, the core layer K is formed from the steel grade CoCr28Mo6 having a carbon content less than 0.055%, whereas the two cover layers S1, S2 are formed from the titanium alloy Ti-Al6-Nb7 or from the titanium alloy Ti-6Al-4V, wherein the titanium alloy that is used is present in the strain-hardened state. According to a third alternative example embodiment, the core layer K is formed from the steel grade CoCr28Mo6 having a carbon content less than 0.055%, whereas the two cover layers S2, S2, are formed from the steel grade X2CrMnMo 18-13-3. According to a fourth alternative example embodiment, the core layer K is formed from the steel grade X2CrNiMo18-15-3, whereas the cover layers S1, S2 are formed from the steel grade X2CrMnMo 18-13-3.

The present invention is not limited to osteosynthesis plates P for the proximal humerus. Rather, the multilayer design according to example aspects of the invention can be used with different osteosynthesis plates P, such as for application on the distal radius, on the femur, on the cranium, or on the thorax. The thickness of the core layer K and of the cover layers S1, S2 described in the first exemplary embodiment is also regarded only as an example.

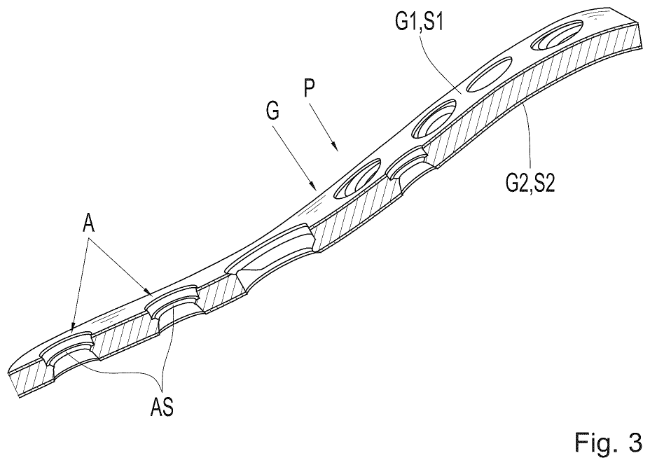

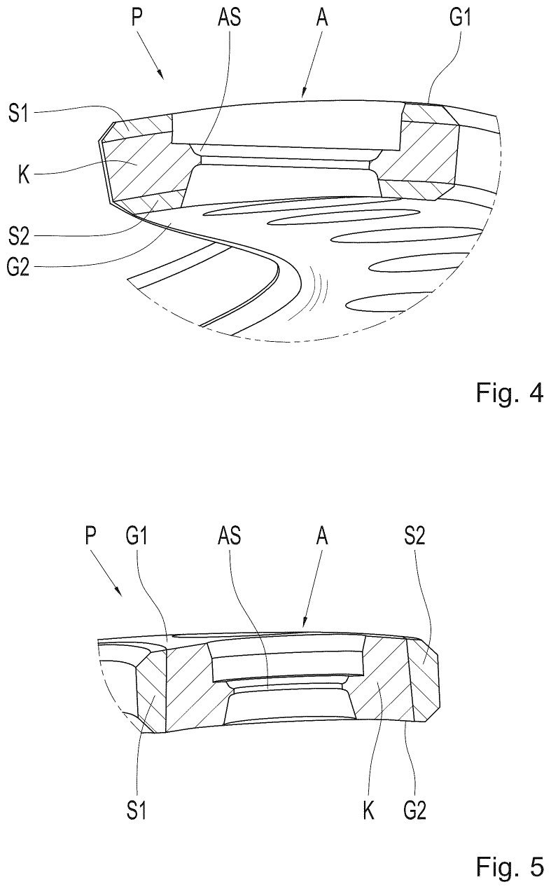

FIG. 3 and FIG. 4 each show a sectional view of a portion of the osteosynthesis plate P according to the first exemplary embodiment. In FIG. 3, a section along a longitudinal extent of the osteosynthesis plate P is represented. In FIG. 4, a section transverse to the longitudinal extent of the osteosynthesis plate P is represented. In both representations, it is clearly apparent that the shoulder AS of one of the openings A is arranged completely in the core layer K. As is clearly apparent in FIG. 4, the arrangement of the shoulder AS can be oblique to a direction of extent of the core layer K, such that a desired pre-alignment of a screw can be achieved. The geometry of the shoulder AS represented in FIG. 4 is regarded only as an example. For clarification, it is pointed out that it is not necessary for a shoulder of this type to be arranged in each opening in the osteosynthesis plate P between the top side G1 and the underside G2.

FIG. 5 shows a section through an osteosynthesis plate P according to a second exemplary embodiment. In contrast to the first exemplary embodiment represented in FIG. 1 through 4, the cover layers S1, S2 extend along opposing lateral faces of the osteosynthesis plate P. The core layer K and the two cover layers S1, S2 extend from the top side G1 to the underside G2. The shoulder AS of the opening A is arranged completely in the core layer K in this example embodiment as well. The layer thickness of the cover layers S1, S2 in this example embodiment is approximately three millimeters (3 mm), measured from the respective lateral face of the osteosynthesis plate P to the core layer K.

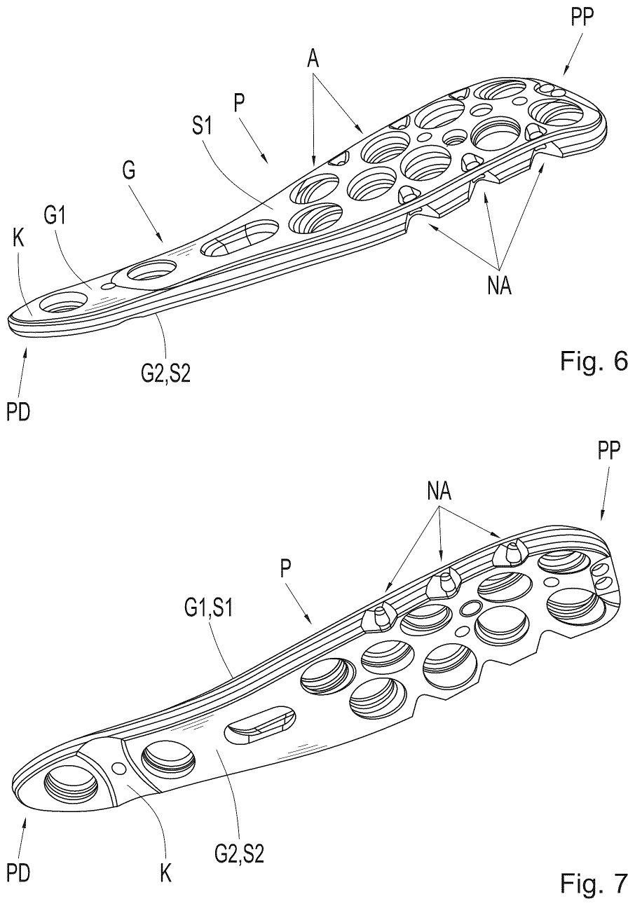

FIG. 6 and FIG. 7 each show an isometric view of an osteosynthesis plate P according to a third exemplary embodiment. In contrast to the first exemplary embodiment represented in FIG. 1 through 4, the cover layers S1, S2, formed on the top side G1 and the underside G2, have a non-constant layer thickness. In the exemplary embodiment represented, the core layer K is at least partially exposed in the region of the distal end PD on the top side G1 and on the underside G2. In other words, the top side G1 and the underside G2 of the osteosynthesis plate P according to the third exemplary embodiment are not covered over the entire surface by the first and the second cover layer S1, S2, respectively. As a result, the distal region PD can be more easily plastically deformed in order to be able to adapt the osteosynthesis plate P at this point to the individual anatomy in the best manner possible. The locally reduced thickness of the cover layers S1, S2 and the local exposure of the core layer K on the top side G1 and the underside G2 are obtained by corresponding machining at these points.

A plurality of suture anchors NA is formed on the side edges of the proximal section PP, for example, to be able to suture tendons or muscles to the osteosynthesis plate P. The suture anchors NA are formed by openings between the top side G1 and the underside G2. These openings are arranged exclusively in the first cover layer S1 in the example embodiment according to the third exemplary embodiment. Recesses are arranged in the core layer K and in the second cover layer S2 in order to make the suture anchors NA more accessible when the osteosynthesis plate is placed with the underside G2 on the bone.

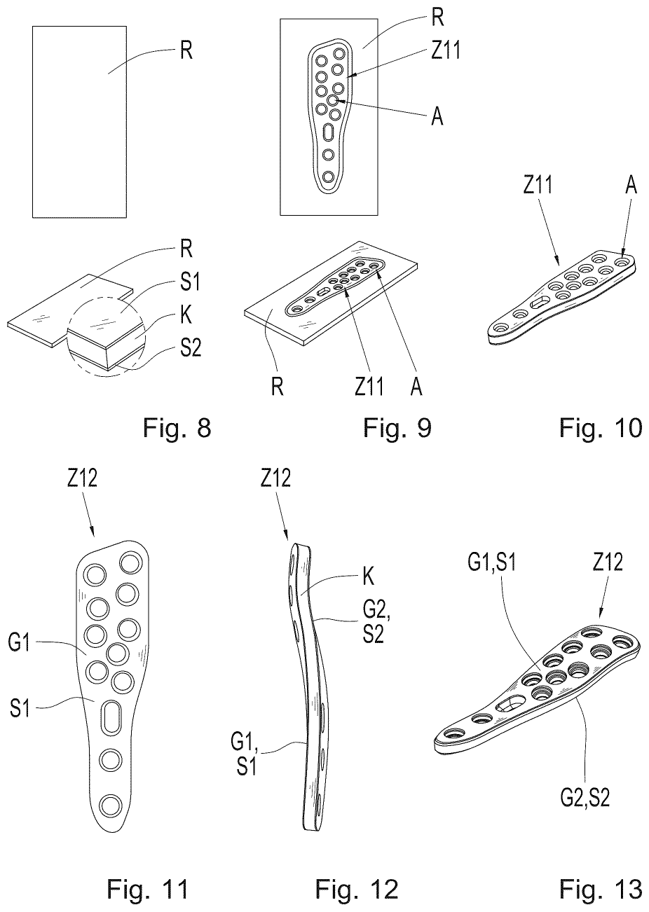

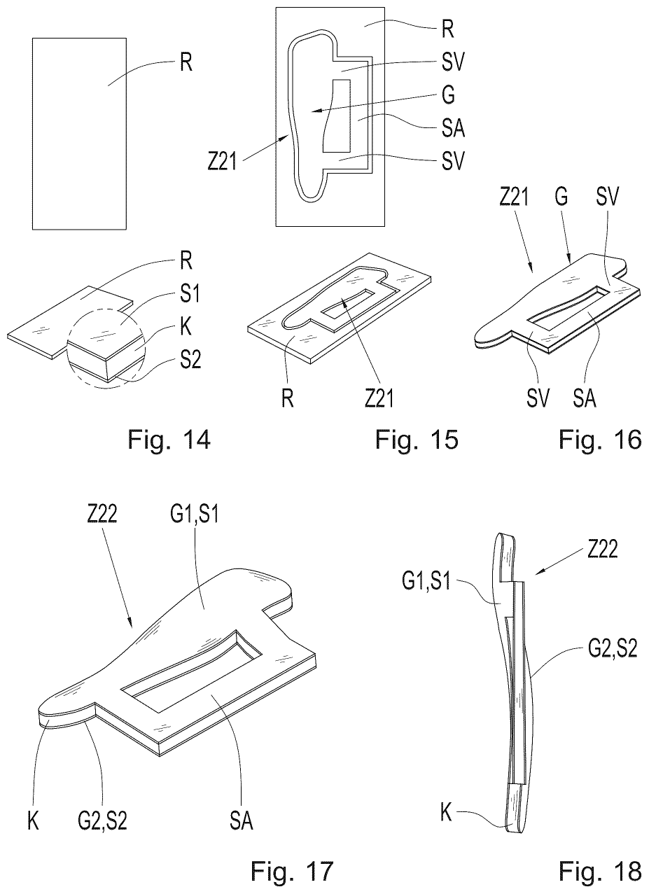

In FIG. 8 through FIG. 22, different methods for manufacturing the osteosynthesis plate P are described by representing intermediate stages of the osteosynthesis plate P during the manufacture. FIG. 8 through FIG. 13 show intermediate stages according to a first example manufacturing method, and FIG. 14 through FIG. 22 show intermediate stages according to a second example manufacturing method.

FIG. 8 shows a top view of a planar raw material R in the upper representation, and an isometric view of the raw material R in the lower representation, wherein a corner of the raw material R is shown in an enlarged representation. The raw material R already has the multilayer design with the core layer K as well as the first and the second cover layer S1, S2. In a first method step for manufacturing the osteosynthesis plate P, a raw material R of this type is provided.

FIG. 9 shows a top view of the planar raw material R in an upper representation, from which a planar intermediate stage Z11 has been separated. The lower representation of FIG. 9 shows a corresponding isometric view. In a second method step for manufacturing the osteosynthesis plate P, this type of separation of the intermediate stage Z11 from the raw material R is carried out. The planar intermediate stage Z11 can be separated out, for example, by water-jet cutting or laser cutting. The planar intermediate stage Z11 already has the openings A. The openings A can already be done and available in the intermediate stage Z11. Alternatively, the openings A in the intermediate stage Z11 can only be preformed, and are further processed into the final shape in a later method step. The openings A in the intermediate stage Z11 are fabricated, for example, by drilling and/or milling. The openings A can be fabricated prior to or after the separation of the intermediate stage Z11 from the raw material R.

FIG. 10 shows the planar intermediate stage Z11 after removal from the raw material R. In this representation, it is clearly apparent that the intermediate stage Z11 is planar, i.e. does not yet have the desired non-planar shape of the osteosynthesis plate P.

FIG. 11 through FIG. 13 show different views of a non-planar intermediate stage Z12 of the osteosynthesis plate. This non-planar intermediate stage Z12 is produced in a third method step for manufacturing the osteosynthesis plate P by shaping the planar intermediate stage Z11. As is clearly apparent in the representations in FIG. 11 through FIG. 13, the layered design is retained during the shaping of the planar intermediate stage Z11 to form the non-planar intermediate stage Z12.

In a fourth method step for manufacturing the osteosynthesis plate P, a heat treatment of the non-planar intermediate stage Z12 is carried out. Since the outer shape of the intermediate stage Z12 does not noticeably change in the process, the intermediate stage of the osteosynthesis plate P after this heat treatment is not represented in the figures. Due to the heat treatment, stresses in the non-planar intermediate stage Z12 induced by the shaping are reduced. Thus, the cover layers S1, S2 are prevented from detaching from the core layer K later.

In an additional method step after the heat treatment, chamfers are fabricated on the edges, such that the osteosynthesis plate P represented in FIG. 1 and FIG. 2 is manufactured. In addition, a surface treatment and/or a surface coating of the osteosynthesis plate P can be carried out, for example grinding, shot peening, or anodization.

FIG. 14 shows a top view of a planar raw material R in the upper representation, and an isometric view of the raw material R in the lower representation, wherein a corner of the raw material R is shown in an enlarged representation. The raw material R already has the multilayer design with the core layer K as well as the first and the second cover layer S1, S2. In a first method step for manufacturing the osteosynthesis plate P, a raw material R of this type is provided.

FIG. 15 shows a top view of the planar raw material R in an upper representation, from which a planar intermediate stage Z21 has been separated. The lower representation of FIG. 15 shows a corresponding isometric view. In a second method step for manufacturing the osteosynthesis plate P, this type of separation of the intermediate stage Z21 from the raw material R is carried out. The planar intermediate stage Z21 can be separated out, for example, by water-jet cutting or laser cutting. The intermediate stage Z21 has the main body G of the osteosynthesis plate P and a clamping section SA, which is connected to the main body G via two connecting segments SV. In contrast to the intermediate stage Z11 represented in FIG. 9, the intermediate stage Z21 does not have any openings in the main body G.

FIG. 16 shows the planar intermediate stage Z21 after removal from the raw material R. In this representation, it is clearly apparent that the intermediate stage Z21 is planar, i.e. does not yet have the desired non-planar shape of the osteosynthesis plate P.

FIG. 17 and FIG. 18 each show a view of a non-planar intermediate stage Z22 of the osteosynthesis plate P. This non-planar intermediate stage Z22 is produced in a third method step for manufacturing the osteosynthesis plate P by shaping the planar intermediate stage Z21. As is clearly apparent in the representations in FIG. 17 and FIG. 18, the layered design is retained during the shaping of the planar intermediate stage Z21 to form the non-planar intermediate stage Z22. The clamping section SA is also retained in the non-planar intermediate stage Z22 and can be used during the shaping to counter-hold or grasp the main body G.

In a fourth method step for manufacturing the osteosynthesis plate P, a heat treatment of the non-planar intermediate stage Z22 is carried out. Since the outer shape of the intermediate stage Z22 does not noticeably change in the process, the intermediate stage of the osteosynthesis plate P after this heat treatment is not represented in the figures. Due to the heat treatment, stresses in the non-planar intermediate stage Z22 induced by the shaping are reduced. Thus, the cover layers S1, S2 are prevented from detaching from the core layer K later.

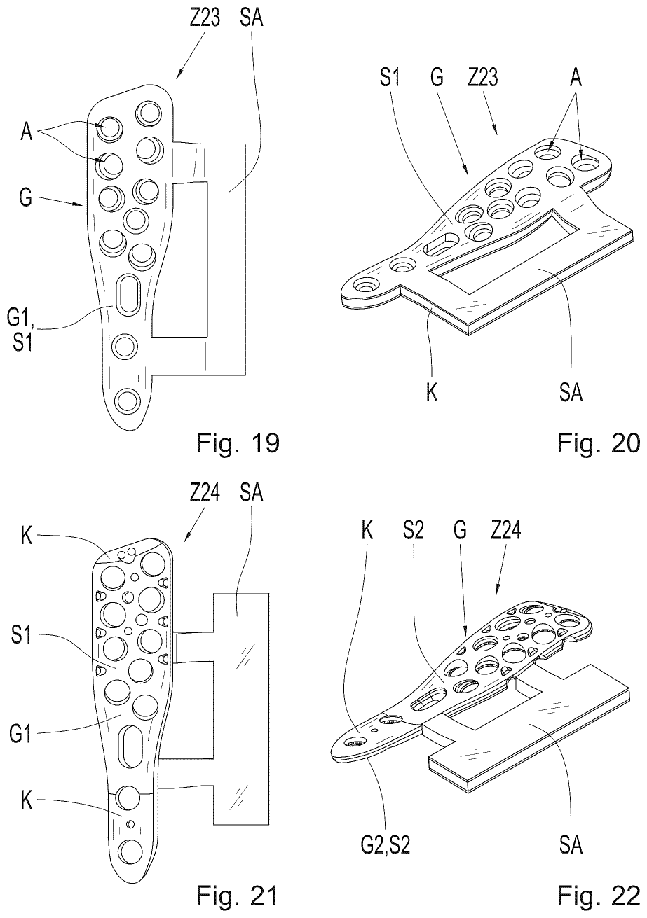

FIG. 19 and FIG. 20 each show a view of an intermediate stage Z23, which has the openings A in the main body G. The openings A are fabricated by machining in a fifth method step for manufacturing the osteosynthesis plate P. The clamping section SA is also retained in the intermediate stage Z23 and can be used to clamp the main body G during machining.

In an additional method step after the fabrication of the openings A, chamfers are fabricated on the edges and the clamping section SA is separated from the main body G, such that the osteosynthesis plate P represented in FIG. 1 and FIG. 2 is manufactured. In addition, a surface treatment and/or a surface coating of the osteosynthesis plate P can be carried out, for example grinding, shot peening, or anodization.

If the objective, however, is to manufacture an osteosynthesis plate P according to the third exemplary embodiment represented in FIG. 6 and FIG. 7, further machining can be carried out prior to the additional method step, which machining removes at least portions of the cover layers S1, S2. An intermediate stage Z24 arising during such machining is represented in FIG. 21 and FIG. 22. The clamping section SA is also retained in the intermediate stage Z24 and can be used to clamp the main body G during machining. Then, chamfers are fabricated on the edges and the clamping section SA is separated from the main body G, such that the end product provided is the osteosynthesis plate P represented in FIG. 6 and FIG. 7.

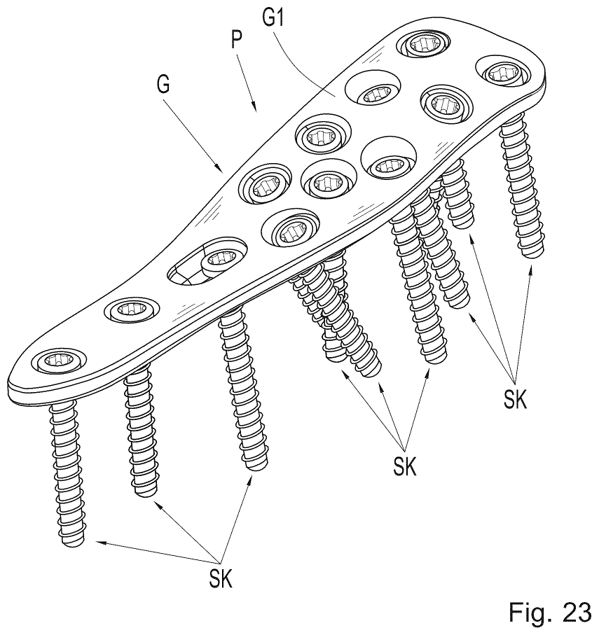

FIG. 23 shows a view of the osteosynthesis plate P according to the second exemplary embodiment represented in FIG. 1 through FIG. 4. In this view, the screws SK are shown, which are arranged in the openings A in the main body G of the osteosynthesis plate P. For clarification, it is pointed out that a screw SK does not need to be arranged in each of the openings A in order to attach the osteosynthesis plate P to the bone.

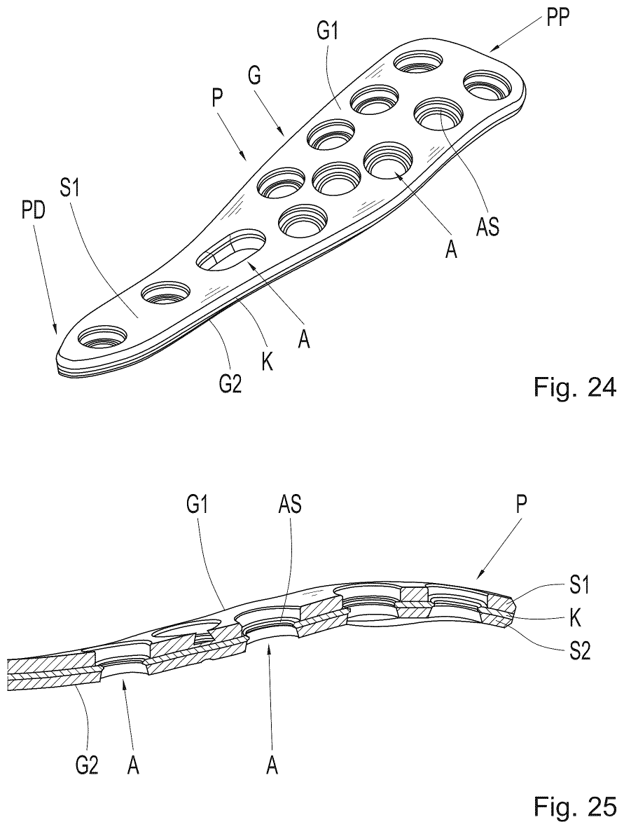

FIG. 24 shows an isometric view of an osteosynthesis plate P according to a fourth exemplary embodiment, which substantially corresponds to the first exemplary embodiment represented in FIG. 1 through 4. The core layer K in this fifth exemplary embodiment, however, is designed to be thinner than the cover layers S1, S2. In the case of such a thin core layer K, in order to be able to arrange the shoulders AS of the openings A completely in the core layer K, the shoulders AS are aligned parallel to the direction of extent of the core layer K.

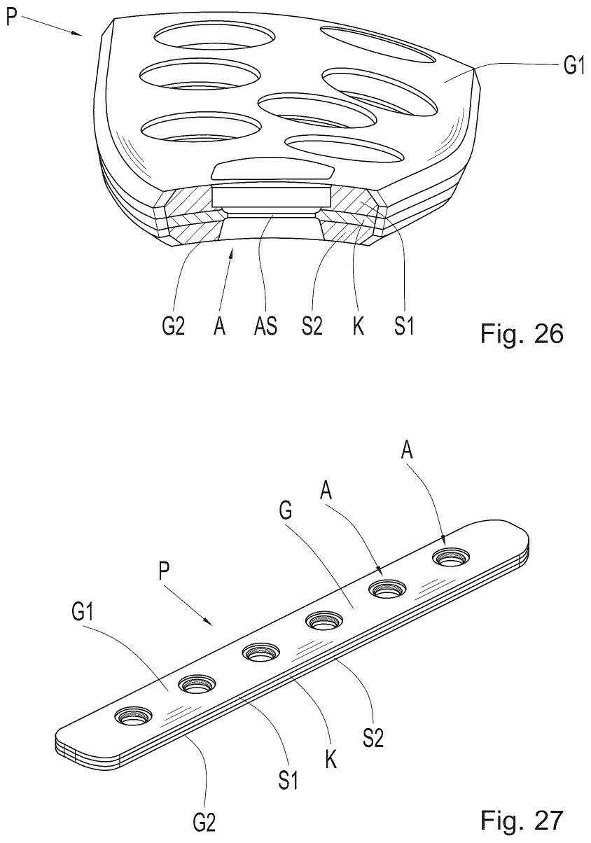

FIG. 25 and FIG. 26 each show a sectional view of the osteosynthesis plate P represented in FIG. 24. In these views, the layer thicknesses of the core layer K and of the cover layers S1, S2 that have been changed in comparison to the first exemplary embodiment represented in FIG. 1 through 4 are clearly apparent. The alignment of the shoulders AS parallel to the direction of extent of the core layer K is clearly apparent in FIG. 25 and FIG. 26.

FIG. 27 shows an isometric view of an osteosynthesis plate P according to a fifth exemplary embodiment, which has a completely different design than that of the above-described exemplary embodiments. The osteosynthesis plate P according to the fifth exemplary embodiment has an elongate main body G with a cross-section that is constant along its direction of extent. The constant cross-section is interrupted only by roundings on the ends and by the openings A, such that an osteosynthesis plate P of this type is relatively easy to manufacture.

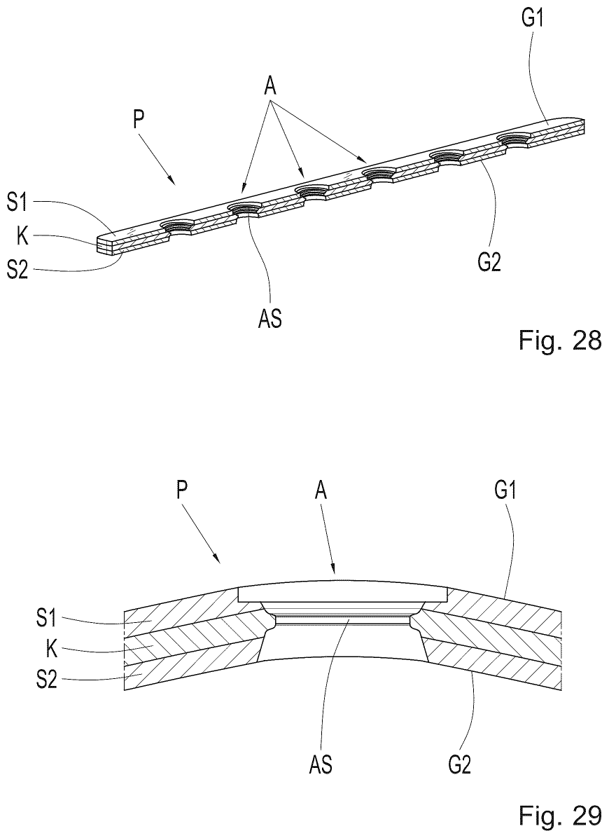

FIG. 28 shows a sectional view of the osteosynthesis plate P represented in FIG. 27. In this sectional view, it is clearly apparent that the core layer K and the cover layers S1, S2 largely have the same layer thickness, which is constant along the longitudinal extent of the osteosynthesis plate P apart from the openings A. The shoulders AS of the openings A are arranged completely in the core layer K by aligning the shoulders AS parallel to the direction of extent of the core layer K.

FIG. 29 shows a further sectional view of the osteosynthesis plate P represented in FIG. 27 and FIG. 28, wherein the sectional plane is aligned transversely to the longitudinal extent of the osteosynthesis plate P. In this further sectional view, it is clearly apparent that the osteosynthesis plate P has a non-planar shape. The osteosynthesis plate P has a concave curvature on the underside G2 in order to ensure a good contact face onto a round bone geometry. In order to obtain a constant cross-section, the top side G1 has a corresponding convex curvature. The geometry of the opening A including the geometry of the shoulder AS is created by machining, such that the layer thickness of the core layer K and of the cover layers S1, S2 is not constant in the region of the openings A. The non-planar shape of the osteosynthesis plate P is produced by shaping a raw material which already has the layered design with the core layer K and the cover layers S1, S2. The geometry of the shoulder AS is created only after the shaping process.

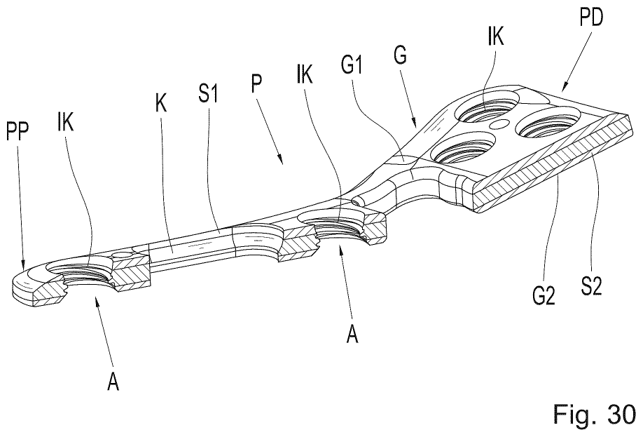

FIG. 30 shows a sectional view of a sixth exemplary embodiment of an osteosynthesis plate P, which is designed to stabilize a distal radius fracture. The distal end PD of the osteosynthesis plate P is to be placed in the region of the wrist joint, whereas the proximal end PP is arranged in the region of the diaphysis of the radius. The osteosynthesis plate P has a core layer K and cover layers S1, S2, each of which has a constant layer thickness. An internal thread IK is formed in each of the openings A in the osteosynthesis plate P, which are designed to hold screws which are not represented in FIG. 30.

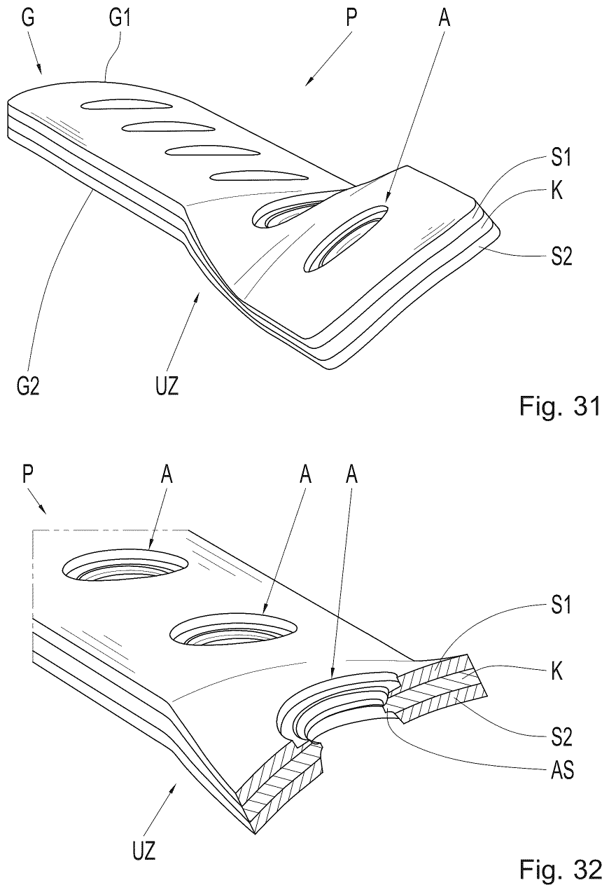

FIG. 31 and FIG. 32 each show a view of an osteosynthesis plate P according to a seventh exemplary embodiment, which, in contrast to the exemplary embodiment represented in FIG. 30, does not have an internal thread in the openings A. Instead, the openings A have a shoulder AS for holding a screw, which is not represented in FIG. 31 and FIG. 32. The osteosynthesis plate P according to the seventh exemplary embodiment has a shaping zone UZ. In this shaping zone UZ, the osteosynthesis plate P is plastically deformed in order to form an anatomically advantageous geometry. The geometry of the openings A including the shoulder AS was introduced prior to the shaping process, such that the shoulder AS, which has been deformed during the shaping process, has a non-rotationally symmetrical shape. This is clearly apparent in the exaggerated shape in the sectional representation according to FIG. 32.

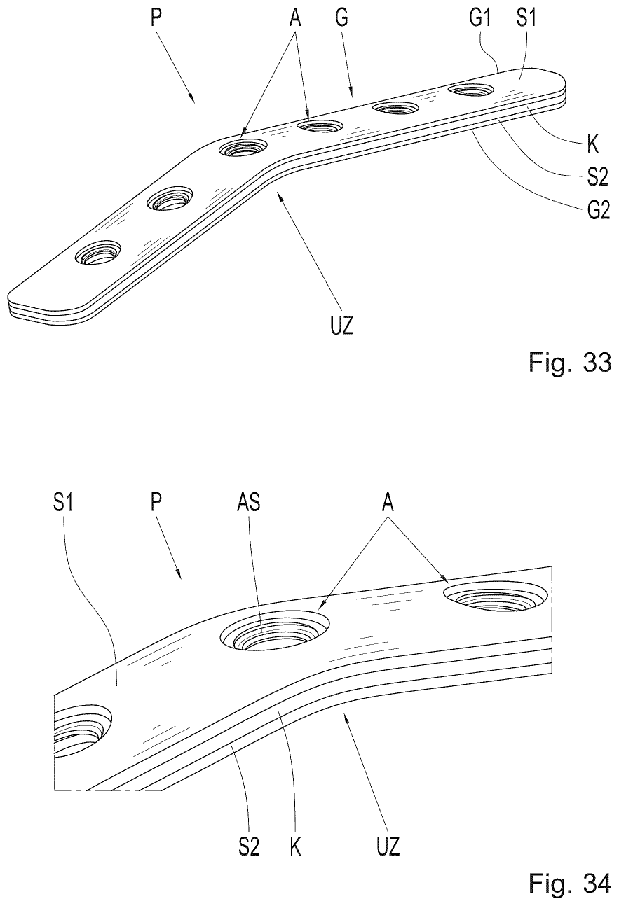

FIG. 33 and FIG. 34 each show a view of an osteosynthesis plate P according to an eighth exemplary embodiment, wherein a shaping zone UZ is present, similarly to the seventh exemplary embodiment represented in FIG. 31 and FIG. 32. The osteosynthesis plate P according to the eighth exemplary embodiment is deformed in only one plane at a point along its longitudinal extent. An additional torsion along the longitudinal extent has not been carried out here. One of the openings A is located in the shaping zone UZ. The geometry of the openings A was introduced prior to the shaping process, such that the shoulder AS, which was deformed during the shaping process, has a non-rotationally symmetrical shape.

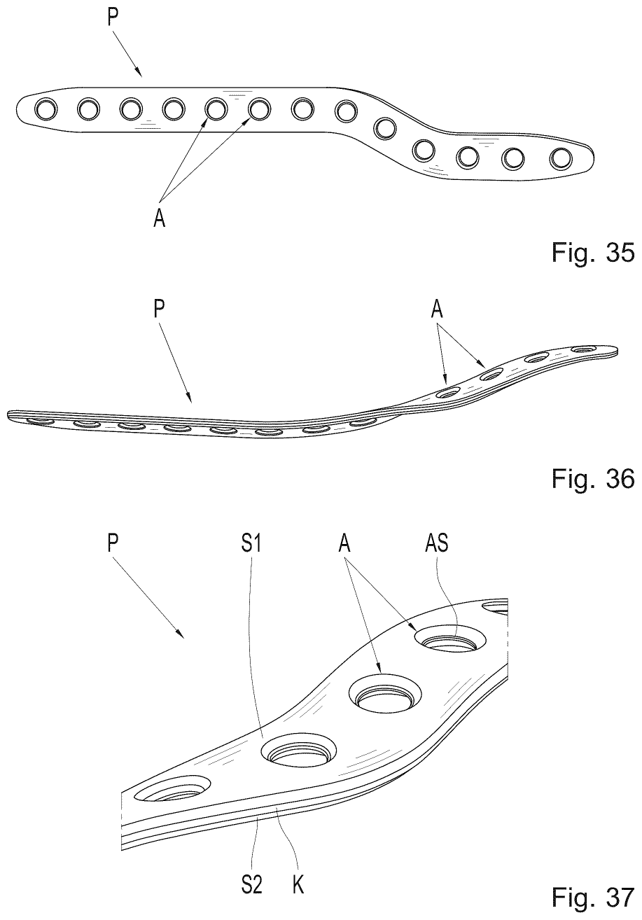

FIG. 35 and FIG. 37 each show a view of an osteosynthesis plate P according to a ninth exemplary embodiment, which has a thinner cross-section than the osteosynthesis plate P according to the seventh and the eighth exemplary embodiment. Thus, the osteosynthesis plate P according to the ninth exemplary embodiment can be three-dimensionally deformed along its entire extent, such that an optimal adaptation to an anatomy is given. The openings A including the shoulder AS are preferably introduced into the osteosynthesis plate P prior to the shaping process. Alternatively, the openings, and/or the geometry of the shoulder AS, can be fabricated only after the shaping process.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

List of reference characters

| | |
|---|---|
| P | osteosynthesis plate |
| PP | proximal end |
| PD | distal end |
| G | main body |
| G1 | top side |
| G2 | underside |
| A | opening |
| AS | shoulder |
| IK | internal thread |
| K | core layer |
| S1 | first cover layer |
| S2 | second cover layer |
| NA | suture anchor |
| SK | screw |
| R | raw material |
| Z11 | intermediate stage |
| Z12 | intermediate stage |
| Z21 | intermediate stage |
| Z22 | intermediate stage |
| Z23 | intermediate stage |
| Z24 | intermediate stage |
| SA | clamping section |
| SV | connecting segments |
| UZ | shaping zone |

1-21: (canceled)

22. An osteosynthesis plate (P) for fixation of bone fragments, comprising:
a main body (G) with a top side (G1) and an underside (G2) disposed opposite the top side (G1), the underside (G2) configured for placement of the osteosynthesis plate (P) on the bone fragments, the main body (G) comprising a plurality of openings (A) formed between the top side (G1) and the underside (G2), each of the plurality of openings (A) configured for holding a respective screw (SK),
wherein the main body (G) has a multilayer construction that comprises a core layer (K), a first cover layer (S1) that at least partially covers the core layer (K) on a first side, and a second cover layer (S2) that at least partially covers the core layer (K) on a second side disposed opposite the first side,
wherein the core layer (K) is formed from a first material, and the first and second cover layers (S1, S2) are formed from a second material, and
wherein the first material differs from the second material, and the first material has greater ductility than the second material.

23. The osteosynthesis plate (P) of claim 22, wherein:
the core layer (K) is formed from pure titanium, and
the first and the second cover layers (S1, S2) are formed from a titanium alloy or from steel.

24. The osteosynthesis plate (P) of claim 22, wherein:
the core layer (K) is formed from grade 1 titanium, grade 2 titanium, grade 3 titanium, or grade 4 titanium; and
the first and the second cover layers (S1, S2) are formed from Ti-6Al-4V, Ti—Al6-Nb7, X2CrNiMo18-15-3, X2CrMnMo 18-13-3, X4CrNiMnMo 21-9-4, or CoCr28Mo6 having a carbon content greater than 0.2%.

25. The osteosynthesis plate (P) of claim 22, wherein:
the core layer (K) is formed from a first steel quality; and
the first and the second cover layers (S1, S2) are formed from a second steel quality or a titanium alloy.

26. The osteosynthesis plate (P) of claim 22, wherein:
the core layer (K) is formed from CoCr28Mo6 having a carbon content less than 0.055%, X2CrNiMo18-15-3, X4CrNiMnMo 21-9-4, or X2CrMnMo 18-13-3; and
the first and the second cover layers (S1, S2) are formed from CoCr28Mo6 having a carbon content greater than 0.2%, X2CrNiMo18-15-3, X4CrNiMnMo 21-9-4, 2CrMnMo 18-13-3, Ti—Al6-Nb7, or Ti-6Al-4V.

27. The osteosynthesis plate (P) of claim 22, wherein a shoulder (AS) for supporting the respective screw (SK) is formed in at least one of the openings (A), and the shoulder (AS) is arranged in the core layer (K).

28. The osteosynthesis plate (P) of claim 22, wherein an internal thread (IK) is formed in at least one of the openings (A), the internal thread (IK) formed either exclusively or predominantly in the core layer (K) and is configured to cooperate with an external thread of the respective screw (SK).

29. The osteosynthesis plate (P) of claim 22, wherein one or more of the core layer (K), the first cover layer (S1), and the second cover layer (S2) has a constant layer thickness.

30. The osteosynthesis plate (P) of claim 22, wherein the first cover layer (S1) has the same layer thickness as the second cover layer (S2).

31. The osteosynthesis plate (P) of claim 22, wherein:
the core layer (K) has a constant layer thickness; and
one or both of the first cover layer (S2) and the second cover layer (S2) has a non-constant layer thickness.

32. The osteosynthesis plate (P) of claim 22, wherein the first cover layer (S1) is formed exclusively on the top side (G1) of the main body (G), and the second cover layer (S2) is formed exclusively on the underside (G2) of the main body (G).

33. The osteosynthesis plate (P) of claim 32, wherein one or both of:
the first cover layer (S1) covers an entire surface of the core layer (K) on the top side (G1) of the main body (G); and
the second cover layer (S2) covers an entire surface of the core layer (K) on the underside (G2) of the main body (G).

34. The osteosynthesis plate (P) of claim 32, wherein a layer thickness of one or both of the first cover layer (S1) and the second cover layer (S2) is no less than two-tenths millimeter and no greater than two millimeters.

35. The osteosynthesis plate (P) of claim 32, wherein one of both of the first cover layer (S1) and the second cover layer (S2) covers the core layer (K) only in sections such that the core layer (K) is at least partially exposed on the top side (G1) and/or on the underside (G2) of the main body (G).

36. The osteosynthesis plate (P) of claim 32, wherein the core layer (K) has a layer thickness that is at least one and a half times as great as that of the first and the second cover layers (S1, S2) at a thickest point of the first and the second cover layers (S1, S2).

37. The osteosynthesis plate (P) of claim 32, wherein the core layer (K) has a layer thickness that is at least three times as great as that of the first and the second cover layers (S1, S2) at a thickest point of the first and the second cover layers (S1, S2).

38. The osteosynthesis plate (P) of claim 36, wherein:
a shoulder (AS) for supporting the respective screw (SK) is formed in at least one of the openings (A);
the shoulder (AS) is arranged in the core layer (K); and
the shoulder (AS) is aligned obliquely to a direction of extent of the core layer (K).

39. The osteosynthesis plate (P) of claim 32, wherein the core layer (K) has a layer thickness that is no greater than one and a half times as great as that of the first and the second cover layers (S1, S2) at a thickest point of the first and the second cover layers (S1, S2).

40. The osteosynthesis plate (P) of claim 39, wherein:
a shoulder (AS) for supporting the respective screw (SK) is formed in at least one of the openings (A);
the shoulder (AS) is arranged in the core layer (K); and
the shoulder (AS) is aligned parallel to a direction of extent of the core layer (K).

41. The osteosynthesis plate (P) of claim 22, wherein:
the first cover layer (S1) and the second cover layer (S2) extend along opposing lateral faces of the osteosynthesis plate (P) such that the core layer (K), the first cover layer (S1), and the second cover layer (S2) extend from the top side (G1) to the underside (G2); and
a layer thickness of one or both of the first and second cover layers (S1, S2) is no less than a half millimeter and no greater than five millimeters.

42. The osteosynthesis plate (P) of claim 22, wherein the osteosynthesis plate (P) has an elongate, non-planar shape.

43. A method for manufacturing the osteosynthesis plate (P) of claim 22, comprising:
providing a planar raw material (R) with the core layer (K) as well as the first and the second cover layers (S1, S2);
separating an intermediate stage (Z11, Z21) of the osteosynthesis plate (P) from the raw material (R);
shaping in order to obtain a desired non-planar shape of the osteosynthesis plate (P); and
heat-treating the non-planar shape of the osteosynthesis plate (P).

44. The method of claim 43, further comprising one or both of surface-treating and surface-coating the heat-treated osteosynthesis plate (P).

45. The method of claim 43, wherein:
the separating the intermediate stage (Z11, Z21) is carried out prior to the shaping such that the intermediate stage (Z11, Z12) separated from the raw material (R) has a planar shape; or
the separating the intermediate stage (Z11, Z21) is carried out after the shaping such that the intermediate stage separated from the raw material (R) has the desired non-planar shape.

46. The method of claim 43, further comprising machining openings (A) of the osteosynthesis plate (P), wherein the machining is carried out immediately after either the separating the intermediate stage (Z11, Z21), the shaping, or the heat-treating.
